(19) United States
(12) Patent Application Publication
(10) Pub. No.: US 2014/0343039 A1
(43) Pub. Date: Nov. 20, 2014

(54) PROCESS FOR PREPARING CEPHALOTAXINE ESTERS

(71) Applicants: Jean-Pierre ROBIN, Geneve (CH); Nina RADOSEVIC, Eguilles (FR); Julie BLANCHARD, Puyricard (FR)

(72) Inventors: Jean-Pierre ROBIN, Geneve (CH); Nina RADOSEVIC, Eguilles (FR); Julie BLANCHARD, Puyricard (FR)

(21) Appl. No.: 14/297,736

(22) Filed: Jun. 6, 2014

Related U.S. Application Data
(62) Division of application No. 13/255,962, filed on Oct. 6, 2011, now abandoned.
(60) Provisional application No. 61/159,186, filed on Mar. 11, 2009.

(51) Int. Cl. C07D 491/056; C07D 307/58; C07D 263/44; C07D 307/60; C07D 498/08

(52) U.S. Cl. CPC C07D 491/056; C07D 307/60; C07D 498/08; C07D 263/44; C07D 307/58
USPC 514/214.01; 540/581; 549/229; 540/546; 548/227; 549/323

(57) ABSTRACT

There is provided a process for preparing cephalotaxine esters corresponding to the following general formula I, which comprises the cephalotaxine backbone, and that can be written as C(R¹)(R²)(XH)COO[CTX], wherein CTX represents the cephalotaxine backbone, being optionally substituted, the process consisting in bringing the corresponding cephalotaxine compound, or salts, isomers or tautomeric forms thereof, which is free or which is in the form of a metal alkoxide CTXOM, into contact with a heterocyclic side chain precursor having both a bifunctional protected (bidentate) and activated (acylating) form of an acid bearing a hydrogenated heteroatom, in the alpha (α) position with respect to the carboxyl group, and corresponding to the following general formula:

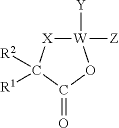

in a customary aprotic solvent, preferably with a catalyst which may be a hindered tertiary amine, at a temperature of between −80° C. and +100° C., preferably in the range 0 to 30° C.

### CROSS-REFERENCE TO RELATED APPLICATIONS

This is a divisional application of U.S. patent application Ser. No. 13/255,962, filed on Mar. 11, 2010 which is U.S. national stage of application no. PCT/IB2010/000835, filed on Mar. 11, 2010, which claims priority to 61/159,186 filed Mar. 11, 2009, the entire disclosures of which are incorporated herein by reference.

### BACKGROUND

Acids and esters bearing a hydrogenated heteroatom in the α position with respect to the carboxyl group are of considerable biological and pharmacological importance and the uses thereof in fine chemistry that are of biological interest are countless [see manual by Coppola et al., in α-Hydroxy Acids in Enantioselective Syntheses Wiley (1997)]. Among them, mention may be made of those which are alpha-hydroxylated, alpha-aminated or alternatively alpha-thiolated. Thus, for example, the esters of the acids, including the block copolymers thereof, constitute peptides and proteins, which form the basis of the chemistry of living organisms.

In nature, there are numerous polycyclic complex molecules belonging to the series of terpene alkaloids or lignans and artificial analogs thereof which are derived from a Darwinian selection process that results in the living beings which secrete them having defenses against non-self (inter alia, various predators, insects, parasitic animals, parasitic fungi, microorganisms, viruses). It should be noted that these poisons “use” all the devices of modern pharmacology to kill this non-self. Chemotherapy in broad senses (anticancer, antiparasitic, antiviral, etc.), resulting from natural substances, is among the most important applications.

One notable fact is that these highly active substances often bear a side chain which, when it is absent, considerably reduces the biological properties of the carrier molecule. Thus, in the plant world, mention may be made of:
terpenes (for example, taxoids or quassinoids, etc.)
lignans (for example, epipodophyllotoxins)
alkaloids, cephalotaxine esters.

For example, among the taxanes, baccatin and 10-deacetyl baccatin have lost all biological activity. The same is true among the harringtonines for these free precursors such as cephalotaxine or drupacine.

Furthermore, since the biosynthesis of these molecules involves the attachment of a precursor of the chain to the main polycyclic molecule, the latter is often abundant in the plant as a hemisynthesis intermediate that is readily available under acceptable economic conditions that are ecologically favorable. The discovery of a method for anchoring a precursor of the side chain bearing a heteroatom, and at least one hindered substituent in the alpha-position with respect to the acid function, on the alcohol function of the naturally available polycyclic molecule is therefore of considerable economic and medical interest. Among these substances, harringtoids, and particularly homoharringtonine, occupy a place of choice since the latter product is on the point of being approved by the United States Food and Drug Administration, as the only drug capable of treating patients having chronic myeloid leukemia and in whom the reference treatment has failed, owing to the appearance of a mutation referred to as T315I. One of our patents (U.S. Pat. No. 6,987,103) described a new method of therapy which use homoharringtonine in the treatment failure of chronic myeloid leukemia by imatinib or dasatinib, the only efficient and commercially available drugs for the therapy of this disease.

More generally, harringtonines are alkaloids which are highly advantageous in cancer chemotherapy and in particular chemotherapy for certain hematosarcomas multiresistant to existing therapeutics. The selectivity of harringtonines is based on a mechanism of intracellular action involving both the inhibition of protein synthesis and the modulation of several signal molecules, which means that, in spite of the recent progress in the protein kinase inhibitor field, this series remain very promising in cancer chemotherapy. Finally the team of this inventor (JPR) have already patented several original processes of anchorage of de side chain on the main core of a number of natural polycyclic moieties as well as in the podophyllotoxin series (U.S. Pat. No. 5,386,016, U.S. Pat. No. 5,643,885 and U.S. Pat. No. 6,107,284) or their analogues than in the taxanes series (U.S. Pat. No. 6,180,802, U.S. Pat. No. 6,285,587, U.S. Pat. No. 6,825,365, U.S. Pat. No. 7,220,871, U.S. Pat. No. 7,279,586 and U.S. Pat. No. 7,432,383) and their analogues or alternatively in the series of alkaloids of this invention, including their unnatural analogues. Economical importance of these inventions may be illustrated by the discovering of tafluposide (a second generation etoposide), the manufacturing of semi-synthetic paclitaxel for pharmaceutical industry at the 100 kilos scale and finally the manufacturing of semi-synthetic alkaloid homoharringtonine (omacetaxine) at the industrial scale.

### DEFINITION

### See Scheme 1

Cephalotaxanes: Cephalotaxanes are alkaloids which have a backbone corresponding to formula F1. This backbone may comprise various oxygenated substituents such as alcohols, phenols and ethers thereof, including those bridged together (internal ethers).

Cephalotaxines: Cephalotaxines are cephalotaxanes which have a secondary alcohol function in the 3-position with respect to the cephalotaxane backbone and which correspond to formula F2.

Cephalotaxine: Cephalotaxine is a cephalotaxine corresponding to formula F3.

Drupacine: Drupacine is a cephalotaxine corresponding to formula F4.

Harringtonines: Harringtonines are cephalotaxines or drupacines esterified in the 3-position by 1-alkylmalic methyl hemiesters, i.e. mixed methyl and cephalotaxyl or drupacyl 1-alkylmalates, corresponding to formula F5 in which X is an oxygen, R2 is anything and R1 is —CH₂CO₂Me.

Isoharringtonines: Isoharringtonines are cephalotaxines or drupacines esterified in the 3-position by 1-alkyl-tartaric methyl hemiesters, i.e. mixed methyl and cephalotaxyl or drupacyl 1-alkyltartrates, corresponding to formula F5 in which X is an oxygen, R2 is anything and R1 is —CHOHCO₂Me.

Harringtoids: Harringtoids are cephalotaxines esterified in the 3-position by a dialkylglycolic acid, corresponding to formula F5 in which X is an oxygen, sulfur or nitrogen atom, R2 is anything and R1 is neither a —CH₂CO₂R radical nor a —CH₂OHCO₂R radical. Harringtoids are artificial substances.

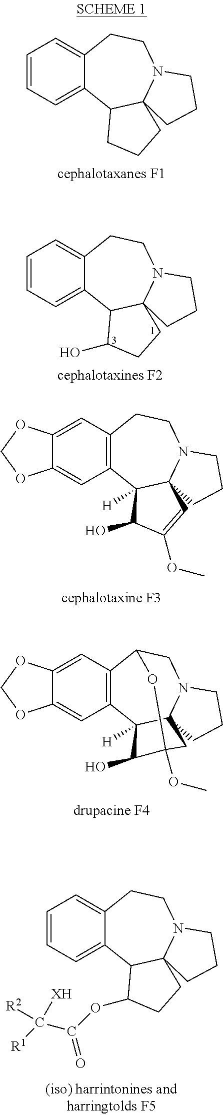

The cephalotaxine esters subject of present invention, can be represented by the following formula, C(R¹)(R²)(XH)COO[CTX],

wherein CTX represents the cephalotaxine backbone, being optionally substituted and/or dehydrogenated. Being not limited to, CTX is preferably selected from the backbone of anyone of the above cephalotaxines having formula F1, F2, F3 or F4.

### DESCRIPTION OF THE PRIOR ART

In the case of the synthesis of the cephalotaxine esters, the subject of the present invention, numerous fruitless attempts were first described, including attempts by carrying out a monofunctional protection of the hydroxyl located in the alpha (α) position with respect to the acid function. Most recently, some laborious attempts have been successfully made, but they were limited to the following particular cases:
The esters do not bear an alcohol, amine or thiol function in the alpha-position with respect to the ester function.
The esters are not hindered.
The cephalotaxine no longer has its alkaloid properties (the authors noted a failure if the nitrogen remained alkaline).

Natural esters of cephalotaxines all exhibit these difficulties simultaneously:
a hydroxyl in the α-position with respect to the carboxyl function,
two encumbered branches in the α-position with respect to the acid function,
a tertiary hydroxyl which has a spontaneous tendency to react with the carboxyl ion necessary for esterification so as to form an α-lactone.

Added to these difficulties is the steric hindrance of the hydroxyl, on the cephalotaxine side.

Thus, the synthesis of deoxyharringtonine hereinafter

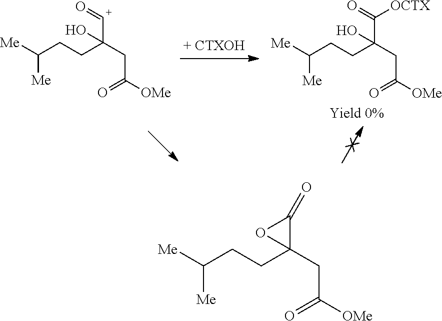

has to date been found to be impossible (at the very least by direct esterification).

The two favorable cases described to date in the literature have been carried out on hydroxylated side chains bearing another function making it possible to protect the hydroxyl located in the alpha-position with respect to the ester bond, by means of a heteroatom bridge.

In the first case, the sensitive tertiary hydroxyl forms a tetrahydropyranyl bridge with the other tertiary hydroxyl (J P Robin et al., Tetrahedron Letters 40 (1999) 2931-2934).

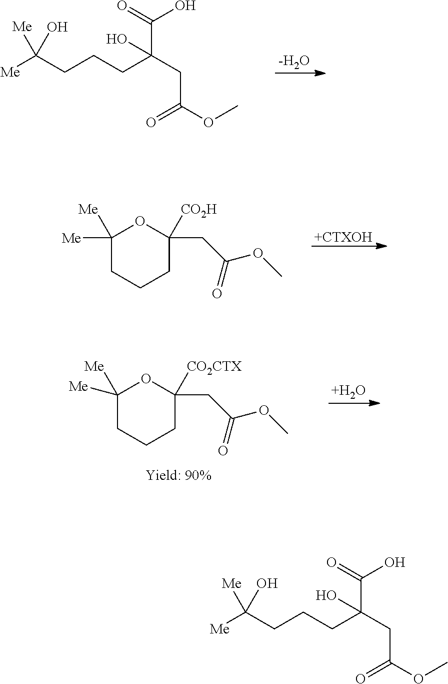

In the second case, for lack of a second tertiary hydroxyl, D Y Gin et al., J. Am. Chem. Soc. (2006) 128, 10370-10371, produced a 4-membered heterocycle (β-lactone) with the hydroxyl of the carboxylate of CH₂CO₂H:

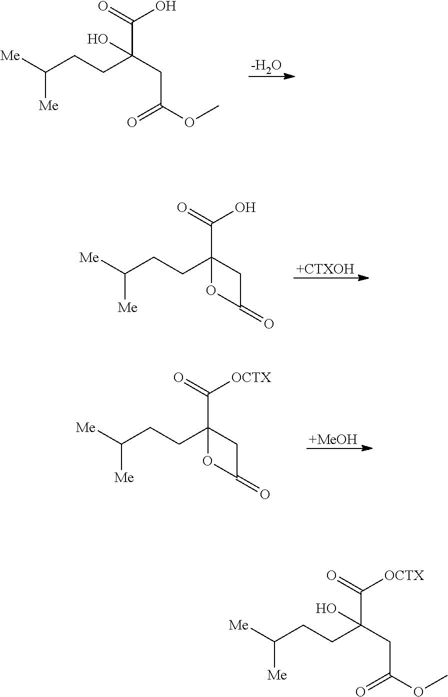

Although the existing syntheses represent progress, they have the following drawbacks:
The synthesis of these cyclic precursors is laborious to carry out.
The regeneration of the linear chain requires drastic conditions that may result in side reactions, requiring further purifications, or may even prove to be impossible to carry out without destroying the resulting molecule.
Especially, these two synthesis methods apply only to particular cases, for example none of these syntheses can apply to the following general case:

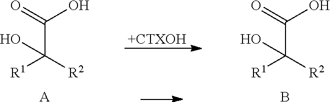

if neither R¹ nor R² contains a substituent capable of being reversibly bridged with the tertiary hydroxyl.

However, since it has been demonstrated that minor variations are capable of considerably modifying the cytostatic activity of these substances, it is important to be able to have a general method of direct synthesis of B, with any R¹ and R² other than in the particular cases mentioned above. Furthermore, since it has been demonstrated that this tertiary hydroxyl, both by virtue of its presence and by virtue of its stereochemistry, plays an essential role in the manifestation of the pharmacological activity, it is also therefore important to have a method of synthesis that also makes it possible to introduce, on this occasion, heteroatoms other than oxygen at this position.

### DESCRIPTION OF THE INVENTION

The present invention comprises a method of efficient esterification of the sterically hindered secondary alcohol function of cephalotaxines, using acids which are themselves hindered and bear a hydrogenated heteroatom (for example an alcohol or an amine or a thiol) in the α-position with respect to the acid function. The principle of this process consists in simultaneously protecting the tertiary hydrogenated heteroatom (OH, SH or NH₂) and the hydroxyl of the acid function with a bifunctional group appropriately chosen so as to activate the acylating properties of the carbonyl in such a way as to form an appropriately substituted heterocycle, according to a known general method of the prior art.

This method has the advantage of applying to chain precursors that are greatly hindered and lack a second function capable of allowing the bifunctional protection, mentioned above.

This synthesis process is described below (see also scheme 2, subsequent page):

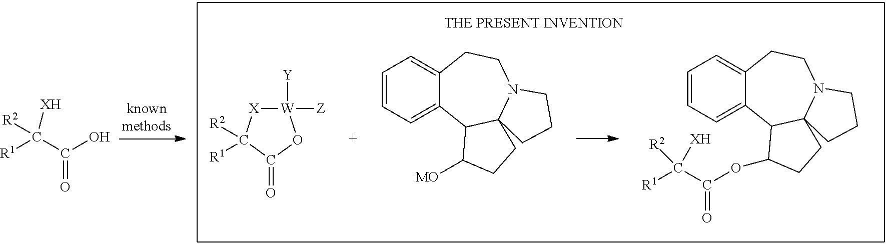

“α-heteroacid”→A+CTXOM→B

The acylating substituted heterocycle A, or isomers, tautometers or salts thereof,

in which W is a carbon, sulfur or silicon atom, X is a heteroatom, preferably an oxygen, a sulfur or a nitrogen, Y and Z are alkyl or heteroalkyl radicals, or monovalent heteroatoms, which may be identical or different, in an independent manner, or may fuse so as to give a divalent heteroatom, it being possible for the X—W bond to be covalent or ionic, and R¹ and R², taken separately, may be alkyl, cycloalkyl, heteroalkyl, aryl, heteroaryl, heterocycloalkyl or aralkyl groups or groups that can form a ring or a heterocycle together,
is brought into contact with the hydroxylated cephalotaxane CTXOM, or isomers or tautomers thereof,
in which M is a hydrogen or a metal, preferably an alkali metal, in a customary aprotic solvent, preferably with a catalyst which may be a hindered tertiary amine, at a temperature of between −80° C. and +100° C., preferably in the range 0 to 30° C.,
so as to give a cephalotaxoid of general formula B, in which X, R¹ and R² have the same meaning as above, in the knowledge that, on the one hand, CTXOH being defined as the cephalotaxine, when X is an oxygen, R¹ is a CH₂CO₂R radical or a CH₂OHCO₂R radical (“iso” series), R is a methyl radical and R² is an optionally hydroxylated alkyl radical, with a hydrocarbon-based backbone taken from benzyl (neoharringtonine), isobutyl (nordeoxyharringtonine), isopentyl (deoxyharringtonine) or isohexyl (homodeoxyharringtonine), we are dealing with the series of harringtonines or isoharringtonines of natural origin and that, on the other hand, when R is other than H or Me, we are dealing with the series of semi-synthetic harringtonines also already described in inventions No. U.S. Pat. No. 6,613,900 (prior. Mar. 30, 1998), U.S. Pat. No. 6,579,869, U.S. Pat. No. 6,831,180, U.S. Pat. No. 7,169,774 and U.S. Pat. No. 7,285,546 (all by Robin et al.)

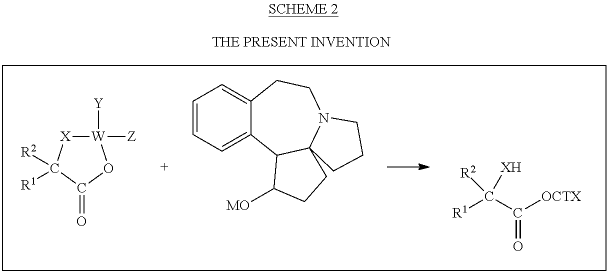

### SELECTION OF EXAMPLES

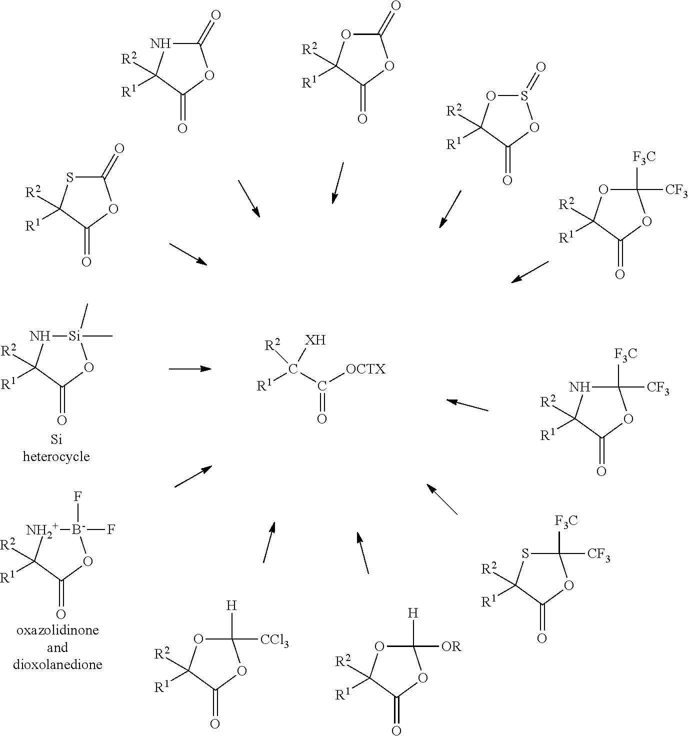

Surprisingly, this method was found to be efficient in the case of alkaloids of the cephalotaxane group, whereas thus far, this process had failed.

The present invention further consists in describing novel compounds belonging to the family of harringtoids corresponding to formula B, in which X, R¹ and R² have the same meaning as above, with the exception of the natural series, already known, for which R¹ is a CH₂CO₂R (harringtonines) or CH₂OHCO₂R (semi-synthetic harringtonines) group, and also the anticancer property thereof and the ability thereof to be part of the composition of a preparation for pharmaceutical or veterinary purposes.

In accordance with the present invention, the following terms encompass the definitions given below.

An alkyl group refers to an aliphatic group that is branched or unbranched and is a saturated hydrocarbon group, having preferably 1 to 24 carbon atoms, such as methyl, ethyl, n-propyl, isopropyl, n-butyl, isobutyl, t-butyl, pentyl, hexyl, heptyl, octyl and the like.

An heteroalkyl group refers to an alkyl group as defined above, wherein at least one of the carbon atom is substituted with a heteroatom such as nitrogen, oxygen, sulfur.

A cycloalkyl group refers to a non-aromatic carbon-based ring having at least three carbon atoms, such as cyclopropyl, cyclobutyl, cyclopentyl, cyclohexyl and the like.

An heterocycloalkyl group refers to an cycloalkyl group as defined above, wherein at least one of the carbon ring is substituted with a heteroatom such as nitrogen, oxygen, sulfur.

An aryl group refers to any carbon-based aromatic group such benzyl, naphtyl. The term “aromatic” includes heteroaryl group which refers to an aromatic group having at least one heteroatom, such as nitrogen, oxygen, sulfur, incorporated within the ring of the aromatic group.

An aralkyl group refers to an aryl group as defined above, having an alkyl group as defined above.

The different subjects of the present invention are the following ones:

A process for preparing cephalotaxine esters corresponding to the following general formula I which comprises the cephalotaxine backbone:

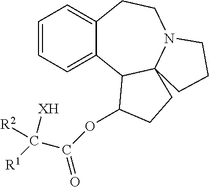

that can also be written C(R¹)(R²)(XH)COO[CTX]
wherein CTX represents the cephalotaxine backbone, being optionally substituted and/or dehydrogenated,
in which formula I, X is a heteroatom, preferably an oxygen, a sulfur or a nitrogen, R¹ and R², taken separately, may be alkyl, cycloalkyl, heteroalkyl, aryl, heteroaryl, heterocycloalkyl or aralkyl groups, said groups being optionally interrupted by ester functions, or groups that can form one or more rings or a heterocycle together,
consisting in bringing the corresponding cephalotaxine compound, or salts, isomers or tautomeric forms thereof, which is free or which is in the form of a metal alkoxide corresponding to the following general formula II:

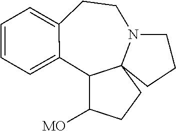

that can also be written CTXOM,
wherein CTX represents the cephalotaxine backbone, being optionally substituted and/or dehydrogenated,
in which M is a hydrogen atom or a metal atom,
into contact with a heterocyclic side chain precursor having both a bifunctional protected (bidentate) and activated (acylating) form of an acid bearing a hydrogenated heteroatom, in the alpha (α) position with respect to the carboxyl group, and corresponding to the following general formula:

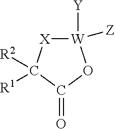

in which case W is a carbon, sulfur, silicon or bore atom, X, R¹ and R² have respectively the same meaning as above, it being possible for R¹ and R² to form a ring or a heterocycle together, and Y and Z are alkyl or heteroalkyl radicals, or monovalent heteroatoms, which may be identical or different, in an independent manner, or may fuse so as to give a divalent heteroatom, it being possible for the X—W bond to be covalent or ionic, in a customary aprotic solvent, preferably with a catalyst which may be a hindered tertiary amine, at a temperature of between −80° C. and +100° C., preferably in the range 0 to 30° C.

In accordance with this process, the following embodiments optionally combined are encompassed:
X may be selected from an oxygen atom, a hydronitrogen (NH) pseudo atom and a sulfur atom,
W is a carbon atom,
Y, Z together fuse to give an oxygen atom,
Y, Z are each an electro-attractive hetero-hydrocarbon group,
Y, Z are identical and are trifluoromethyl,
W is a sulfur atom, X is an oxygen atom and Y, Z together fuse to give an oxygen atom,
W is a silicium atom, X is an oxygen atom and Y, Z are alkyl, aryl or aralkyl group,
R¹ and R² are identical,
R² is —CH₂COO—R³ in which R³ is selected from alkyl, cycloalkyl, heteroalkyl, aryl, heteroaryl, heterocycloalkyl and aralkyl groups, said groups being optionally interrupted by ester functions.

In accordance with the present invention, the following compounds are encompassed:
Compounds having the structural formula I which comprises the cephalotaxine backbone:

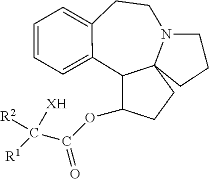

that can also be written C(R¹)(R²)(XH)COO[CTX]
wherein CTX represents the cephalotaxine backbone, being optionally substituted and/or dehydrogenated,
in which X is a heteroatom, preferably an oxygen, a sulfur or a nitrogen, R¹ and R², taken separately, are selected from alkyl, cycloalkyl, heteroalkyl, aryl, heteroaryl, heterocycloalkyl and aralkyl groups and groups that can form one or more rings or a heterocycle together,
with the proviso when together X═O and the tetracyle named CTXOH is cephalotaxine or drupacine and R¹═—CH—₂COO—R³,
R² is not (CH₃)₂(CH₂)ₙ— (with n=1 to 4) or R² is not (CH₂)₂COH(CH₂)ₙ₋₁— or R² is not benzyl or phenyl or homobenzyl, R³ is selected from alkyl, cycloalkyl, heteroalkyl, aryl, heteroaryl, heterocycloalkyl and aralkyl groups, said groups being optionally interrupted by ester functions.
Compounds having the structural formula

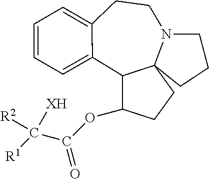

that can also be written C(R¹)(R²)(XH)COO[CTX]
wherein CTX represents the cephalotaxine backbone, being optionally substituted and/or dehydrogenated,
in which X is a heteroatom, preferably an oxygen, a sulfur or a nitrogen, R¹ and R², are identical and may be alkyl, cycloalkyl, heteroalkyl, aryl, heteroaryl, heterocycloalkyl or aralkyl groups or groups that can form one or more rings or a heterocycle together.

Preferred compounds of the present invention are compounds as above defined in which X is an oxygen atom, a nitrogen atom or a sulfur atom.
Compounds having the structural formula

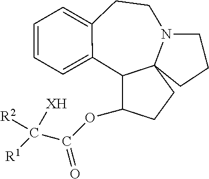

that can also be written C(R¹)(R²)(XH)COO[CTX]
wherein CTX represents the cephalotaxine backbone, being optionally substituted and/or dehydrogenated,
in which X is a nitrogen atom, R¹ and R² are different each other and taken separately, are selected from alkyl, cycloalkyl, heteroalkyl, aryl, heteroaryl, heterocycloalkyl and aralkyl groups and groups that can form one or more rings or a heterocycle together.

Preferred compounds of the present invention are compounds as above defined:
where the tetracyclic core namely the alkaloid moiety is the natural enantiomeric form of cephalotaxine, the later having the below structural formula

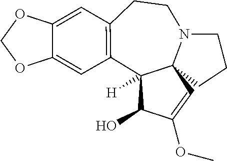

where the tetracyclic core namely the alkaloid moiety is the natural enantiomeric form of drupacine, the later having the below structural formula

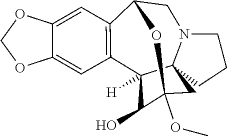

in which the alkaloid moiety is the unnatural enantiomer.

The invention also relates to a subset of compounds having the following structural formula

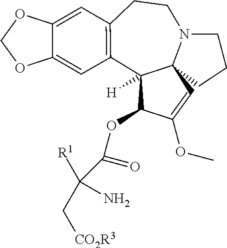

In which R¹ and R² have the same meaning as in claim 1 and R3 has the same meaning as R¹ or R².

More preferred cephalotaxine esters according to the invention are selected from the group consisting of:
(−)-cephalotaxine 2,2-dimethylglycolate, 2a
(−)-cephalotaxine 2,2-diphenylglycolate, 2b
(−)-cephalotaxine 2,2-dibenzylglycolate, 2c
(−)-drupacine 2,2-dibenzylglycolate, 2d
(−)-cephalotaxine 2-aminobutyrate, 2f
(−)-cephalotaxine 2-aminobutyrate, 3c, and
(−)-Cephalotaxine 1-aminocyclohaxane carboxylate, 3d.

The invention also relates to a pharmaceutical preparation comprising one or more of the compounds depicted above. This preparation is designed for treating a disease selected from a human cancer including leukemia, parasitic disease, immune disease or a transplantation rejection.

The invention also concerns a therapeutical method which uses the pharmaceutical preparation as defined above.

Also are encompassed with the present invention, compounds as an intermediary compound for preparing cephalotaxine esters in accordance with the process of the invention, said compound being selected from:
O-carboxyanhydride of diphenylglycolic acid, 1b
O-carboxyanhydride of dibenzyl glycolic acid, 1c
O-carboxyanhydride of primary dimethyl citrate, 1d
O-carboxyanhydride of deoxyharringtonic acid methyl hémiesters, 1e
O-carboxyanhydride of neoharringtonic acid methyl hémiesters, 1f
N-carboxyanhydride of dimethyl glycolic acid, 1g
O—O-Hexafluoroacetonide of methyl citramalic acid hémiesters, 1h
a substituted bis 5,5′-trifluoromethyl-1,4-dioxolanone, 1i
3,3′-dimethyl-5,5′-Bistrifluoromethyl-oxazolidinone, 1j
3,3′-cyclopentylidene-5,5′-Bistrifluoromethyl-oxazolidinone, 1k.

The invention is below explained in detail.

### Preparation of Acylating Heterocycles:

Among these cyclic precursors having acylating properties, mention may be made of diversely substituted dioxolanones (including hexafluorodimethyls), alpha-hydroxy acid O-carboxyanhydrides (or OCOs), alpha-amino acid N-carboxyanhydrides (or NCOs), S-carboxyanhydrides, dialkylsilylidenes, cyclic sulfoxides, more generally cyclic acetals in the broad sense resulting from the bifunctional protection of an acid bearing a hydrogenated heteroatom in the α-position, i.e., for example, of α-hydroxy acids, of α-mercapto acids or of α-amino acids which are organic. The cyclic derivatives have been prepared according to the various methods described in the literature. For example, the alkylmalic acid hexafluoroacetonides have been prepared by the action of hexafluoroacetone on the corresponding appropriately substituted malic acids. The dialkyl carbonylidene glycolates of dialkylglycolic acids or dioxolanedione, also known as O-carboxyanhydrides (OCAs), have been prepared by the action of various phosgene equivalents on the corresponding appropriately substituted free acids. The silylidene derivatives have been prepared by the action of a dialkylsilyl dichloride on the corresponding appropriately substituted free acids. Scheme 3 hereinafter summarizes the various pathways for obtaining the acylating cyclic precursors.

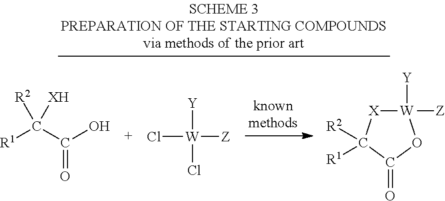

### Selection of Examples

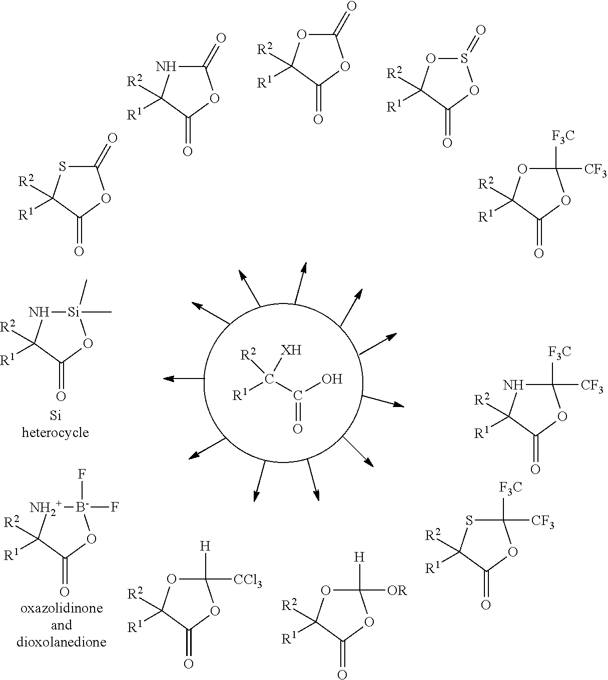

Among the cephalotaxine examples chosen to illustrate the present invention, but without taking away the generality of the scope thereof, we have used cephalotaxine per se or its even more incumbent natural analog drupacine, and also diastereoisomers thereof. These compounds were obtained by chromatographic purification of extracts of various Cephalotaxus.

Among the precursor examples chosen to illustrate the present invention, but without taking away the generality of the scope thereof, we have used the cyclic derivatives of natural chains as well as various derived esters, but with all of these corresponding to the minimum substructure of a dialkyl glycolic acid.

### EXAMPLES

### Example 1

### Preparation of the Intermediate Compounds of Formula

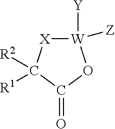

### Examples 1A

### Preparation of O-, N-, S-Carboxyanhydrides of General Formula

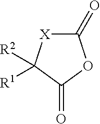

### X═O, N or S

Both of the following general procedures A and B result in the preparation of the above compounds, leading to similar yields, and the illustrated compounds below may be prepared by any one of procedures A and B. Procedure A is however more easily carried out.

### General Procedure A: Disphosgene Method

1.5 equivalents of a solution of triphosgene or diphosgene in tetrahydrofuran (THF), and then 30 g per mol of pulverulent active carbon or of an amine such as pyridine or triethylamine are added dropwise, with care, to a solution or a suspension, stirred at 20° C., under an inert gas, of the alpha-hydroxy acid, of the alpha-amino acid or of alpha-mercapto acid in tetrahydrofuran (THF). The kinetics of the reaction are monitored in the following way: a 100 μL test sample is filtered through glass cotton wool and a drop is placed on the ATR accessory of a Fourier transform infrared spectrograph; the spectrum shows the gradual appearance of the carbonyl band(s) typical of cyclic anhydrides in the 1800-1900 cm⁻¹ range, while the carbonyl band of the free acid function in the 1700-1750 cm⁻¹ range disappears. In the absence of reaction, after one hour, the reaction mixture is brought to the reflux of THF. In most cases, the reaction no longer changes after 12 hours. The reaction mixture is then filtered through a small layer of Celite, so as to give a colorless solution which is evaporated to dryness under reduced pressure. The residue is then triturated from hexane which selectively extracts the cyclic anhydride. The hexane is evaporated to dryness under reduced pressure so as to give a carboxyanhydride that can be used as it is directly in the next stage (the next stage is carried out in the same container). Although generally not isolated here, the carboxyanhydrides are most commonly crystalline and, in certain cases, it has been possible to partially or completely characterize them (IR, NMR). The residue resulting from trituration from hexane is made up of the unchanged starting acid, which can be recovered and recycled. Taking away the recovered acid, the yields are between 60% and 100%, depending on the examples.

### General Procedure B: Carbonyldiimidazole (CDI) Method

1.2 equivalents of CDI are added to a solution or a suspension, stirred at 20° C., under inert gas, of the alpha-hydroxy acid, of the alpha-amino acid or of alpha-mercapto acid in tetrahydrofuran (THF), or dichloromethane. The kinetics of the reaction are monitored in the following way: a 100 μL test sample is filtered through Celite and a drop is placed on the ATR accessory of a Fourier transform infrared spectrograph; the spectrum shows the gradual appearance of the carbonyl band(s) typical of the cyclic anhydrides in the 1800-1900 cm⁻¹ range, while the carbonyl band of the free acid function in the 1700-1750 cm⁻¹ region disappears. After 1 to a few hours, the reaction mixture is chromatographed on silica gel, so as to give a colorless solution which is evaporated to dryness under reduced pressure. In some examples, the alcohol to be esterified can be introduced directly into the reaction medium with the catalyst.

### Example 1a

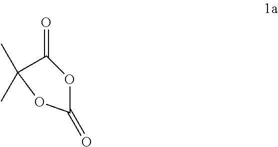

O-carboxyanhydride (“OCA”) of dimethyl glycolic acid Commercially available dimethyl glycolic acid is treated according to the procedure above. The OCA 1a, obtained with a yield of 70-80%, has the following characteristics:

Physical state: pale yellow, thick oil

IR (ATR, film); v2971, 2859, 1892, 1870, 1808, 1780, 1259, 1168, 1064, 988, 905, 764 cm⁻¹.

### Example 1b

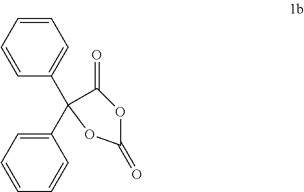 [SMILES: O=C1OC(=O)C(c2ccccc2)(c2ccccc2)O1]

O-carboxyanhydride (“OCA”) of diphenylglycolic acid Commercially available diphenylglycolic acid is treated according to the procedure above. The OCA obtained, 1b, with a yield of 70-80%, has the following characteristics:

Physical state: white crystals

¹H NMR (300 MHz, CDCl₃) δ 7.63-7.32 (m, 10H); ¹³C NMR (75 MHz, CDCl₃) δ 166.90, 147.50, 134.82, 130.39, 129.43, 126.20; IR (neat) ν 3064, 1901, 1871, 1803, 1491, 1449, 1261, 1129, 1018 cm⁻¹.

### Example 1c

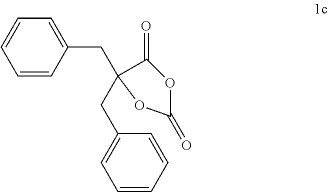 [SMILES: O=C1OC(=O)C(Cc2ccccc2)(Cc2ccccc2)O1]

O-carboxyanhydride (“OCA”) of dibenzyl glycolic acid Commercially available dibenzyl glycolic acid is treated according to the procedure above. The OCA obtained, 1c, has the following characteristics:

Physical state: colorless resin

¹H NMR (300 MHz, CDCl₃) δ 7.52-6.33 (m, 10H), 3.38 (d, J=14.4, 2H), 3.31 (d, J=14.4, 2H); ¹³C NMR (75 MHz, CDCl₃) δ 168.56, 147.16, 135.32, 131.57, 130.39, 129.31, 128.62, 127.40, 91.39, 45.22, 42.10, 30.01; IR (ATR, film, neat) ν 1977, 1809, 1717, 1494, 1453, 1269, 1147, 955 cm⁻¹.

### Example 1d

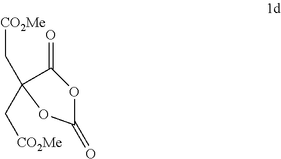 [SMILES: COC(=O)CC1(CC(=O)OC)OC(=O)OC1=O]

O-carboxyanhydride (“OCA”) of primary dimethyl citrate Primary dimethyl citrate obtained according to the process of Hirota et al. [Chemistry Letters, 191 (1980)] is treated according to the procedure above. The OCA 1d, obtained with a yield of 70-80%, has the following characteristics:

Physical state: not isolated

### Example 1e

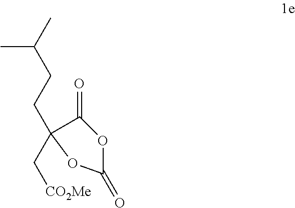 [SMILES: COC(=O)CC1(CCC(C)C)OC(=O)OC1=O]

O-carboxyanhydride (“OCA”) of deoxyharringtonic acid methyl hemiester The highly pure starting deoxyharringtonic acid methyl hemiester was obtained according to a modification of the process of Mikolajczak et al. [Tetrahedron, vol. 28 1995 (1972)] and has the following characteristics:

Formula:

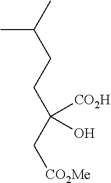

Physical state: Crystalline white solid. Mp 94-95° C.

¹H NMR (300 MHz, CDCl₃) δ 3.71 (s, 3H), 2.99 (d, J=16.6, 1H), 2.74 (d, J=16.7, 1H); 1.82-1.63 (m, OH), 1.52 (tt, J=13.0, 6.6, 1H), 1.39 (ddd, J=17.7, 9.1, 5.5, 1H), 1.12 (ddd, J=18.8, 11.7, 5.7, 1H) 0.89 (d, J=1.0, 3H), 0.87 (d, J=1.1, 3H).

This hemiester was treated according to the general procedure above. The OCA 1e, obtained with a yield of 70-80%, has the following characteristics:

Physical state: colorless resin.

IR (ATR, film, neat), ν 2955, 1881, 1808, 1734, 1439, 1366, 1222, 1067, 971, 892 cm⁻¹.

### Example 1f

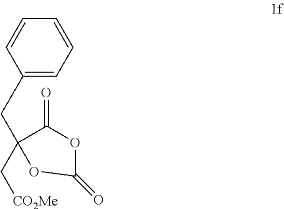 [SMILES: COC(=O)CC1(Cc2ccccc2)OC(=O)OC1=O]

O-carboxyanhydride (“OCA”) of neoharringtonic acid methyl hemiester The highly pure, starting neoharringtonic acid methyl hemiester was obtained according to a modification of the process of Mikolajczak et al. [Tetrahedron, vol. 28 1995 (1972)], and has the following characteristics:

Formula:

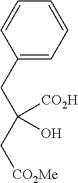 [SMILES: COC(=O)CC(O)(Cc1ccccc1)C(=O)O]

Physical state: crystalline Mp=106-108° C.

¹H NMR (300 MHz, CDCl₃) δ 10.54 (s, 1H), 7.37-7.22 (m, 5H), 3.72 (s, 3H), 3.15-2.96 (m, 3H), 2.75 (d, J=16.6, 1H); IR (KBr) ν 3486, 3031, 1737, 1690, 1441, 1323, 1266, 1203, 1119, 1004, 870 cm⁻¹.

This hemiester was treated according to the general procedure above. The OCA 1f, obtained with a yield of 76%, was not isolated and was directly introduced into the next stage.

### Example 1g

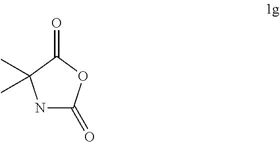

N-carboxyanhydride (“NCA”) of dimethyl glycolic acid Commercially available 2-aminoisobutyric acid (98% purity) is treated according to the procedure above. The NCA 1g, obtained with a yield of 65-75%, exhibited the following characteristics:

Physical state: colorless, thick oil

IR (ATR, film); v2975, 2865, 1880 cm⁻¹.

### Example 1B

### Preparation of Hexafluoroacetonides Intermediates of General Formula

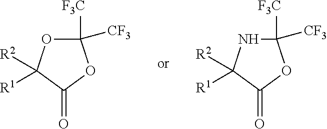

Or, alternatively

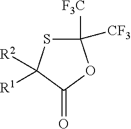

### General Protocol C:

Starting compounds exhibiting below formula are commercially available or are prepared according to the literature description (see also example 1).

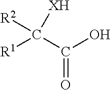

A solution of the above substituted α-hydroxy-, α-mercapto or α-amino acid (4 moles) in 1 L of dimethyl sulfoxide was vigorously stirred under atmosphere of hexafluoroacetone (at least 2 equivalents) at atmospheric pressure, at 20° C.±5. After completion of the reaction, the mixture was quenched with iced-water and the product was extracted with organic solvent (dichloromethane or chloroform) The organic layer was washed with iced-water and dried over magnesium sulfate, then the solvent was evaporated in vacuo and the residue recrystallized from suitable organic solvent or combination of organic solvents. The yielded lactone was enough pure to be used without further purification.

### Example 1h

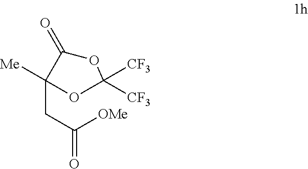 [SMILES: COC(=O)CC1(C)OC(C(F)(F)F)(C(F)(F)F)OC1=O]

### Preparation of O—O-Hexafluoroacetonide (HFA) of Methyl Citramalic Acid Hemiester as Intermediate

Highly purified monomethyl ester of citramalic acid was prepared in using commercial material as starting product. To this purpose, a 0.2 M dichloromethane solution of the citramalic acid was treated at room temperature under vigorous stirring with commercial boron trifluoride-methanol complex. After completion of disappearance of starting diacid (checked by Thin Layer Chromatography=TLC), the reaction was stopped with ice, then the mixture was partitioned between dichloromethane and 5% sodium carbonate. Aqueous sodium salt solution of the hemiester was washed with dichloromethane (3×), then acidified and extracted with dichlormethane. Organic layers were combined and dried over Mg SO4, then evaporated under reduce pressure. This hexafluoroacetonide exhibit the following features:

Formula:

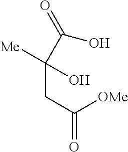 [SMILES: COC(=O)CC(C)(O)C(=O)O]

No trace of the diester or of the other isomer monoester was found (TLC and NMR)

Physic state: White amorphous solid.

Above intermediate was treated according the general protocol C to yield the oxolactone 1h in the 75-85% range yield (1h may be also named as a substituted bis 5,5′-trifluoromethyl 1,4-dioxolanone). Crude product was submitted to flash chromatography (Silicagel, dichloromethane). Evaporation of suitable fractions gave after evaporation a pale yellow foam which was directly uses without further isolation or purification in the cephalotaxine esterification step

Physic state: pale yellow resin.

IR (ATR, film, neat) ν 3400, 1751, 1734, 1435 cm⁻¹.

### Example 1i

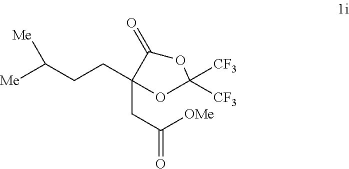 [SMILES: COC(=O)CC1(CCC(C)C)OC(C(F)(F)F)(C(F)(F)F)OC1=O]

Starting methyl hemiesters of deoxyharringtonic acid was prepared according to a modification of the process used in example 1e.

Formula:

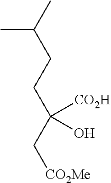

Above intermediate was treated according the general protocol C to yield the lactone 1i (75-85% yield). **Crude product was submitted to flash chromatography (Silicagel, dichloromethane). Evaporation of selected fractions gave after evaporation a white resin which was directly uses without further isolation or purification in the next step

Physic state: white resin.

IR (ATR, film, neat) ν 3405, 1750, 1735, 1433 cm⁻¹.

**1i may be also named as a substituted bis 5,5′-trifluoromethyl-1,4-dioxolanone

### Example 1j

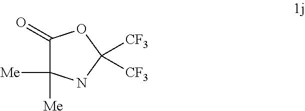 [SMILES: CC1(C)NC(C(F)(F)F)(C(F)(F)F)OC1=O]

3,3′-dimethyl-5,5′-Bistrifluoromethyl-oxazolidinone 1j was prepared in using the general protocol C. Commercially available 2-aminobutyric acid was used as starting material. Crude product was submitted to flash chromatography (Silicagel, dichloromethane). Evaporation of selected fractions gave after evaporation a white resin which was directly used without further isolation or purification in the next step

Physic state: white resin.

IR (ATR, film, neat) ν 3400, 1740, 1430 cm⁻¹.

### Example 1k

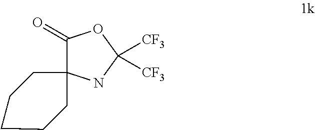 [SMILES: O=C1OC(C(F)(F)F)(C(F)(F)F)NC12CCCCC2]

3,3′-cyclopentylidene-5,5′-Bistrifluoromethyl-oxazolidinone 1k Commercially available 1-aminocyclohexane carboxylic acid was treated according to the general protocol C. used as starting material. Crude product was submitted to medium pressure chromatography (Silicagel Si60, dichloromethane-ethyle acetate). Evaporation of selected fractions (TLC) gave after evaporation a pale yellow glass which was directly used without further isolation in the next step

Physic state: white resin.

IR (ATR, film, neat) ν 3420, 1745 cm⁻¹.

### Example 2

### Preparation of the Cephalotaxyl Substituted Glycolate and Malate Esters* of General Formula

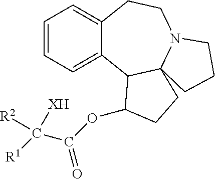

1.5 molar equivalents of the alpha-hydroxy acid carboxyanhydride, of the alpha-amino acid carboxyanhydride or, alternatively, of the alpha-mercapto acid carboxyanhydride in dichloromethane (3 molar equivalents in the case of a racemic product) are added directly in the solid state to a solution, stirred at 20° C., under an inert gas, of a cephalotaxine (1 mol), followed by one equivalent of dimethylaminopyridine, also in the solid state. The progression of the reaction is monitored by thin-layer chromatography and the disappearance of the carbonyl band typical of anhydrides is monitored by IR spectroscopy.

*Malic esters pertained to a special subset of glycolic esters in which R¹ includes the —CH₂CO₂H sidechain. Some of them have special name (i.e. for R₂=methyl the malic acid is called “citramalic acid”)

### Example 2a

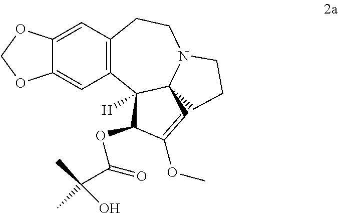

(−)-cephalotaxine 2,2-dimethylglycolate The OCA 1a was brought into contact with purified cephalotaxine according to the general procedure of example 2. The cephalotaxine ester 2a thus obtained has the following characteristics:

Physical state: white powder.

¹H NMR (300 MHz, CDCl₃) δ 6.60 (s, 1H), 6.59 (s, 1H), 5.94-5.80 (m, 4H), 5.07 (s, OH), 3.81 (d, J=9.6, OH), 3.70 (s, 1H), 3.23-3.02 (m, 1H), 3.02-2.89 (m, OH), 2.69-2.55 (m, 1H), 2.37 (dd, J=14.0, 6.6, OH), 2.10-1.67 (m, 2H), 1.20 (s, 3H), 0.82 (s, 3H); IR (KBr) ν 3495, 2941, 2797, 1730, 1655, 1488, 1377, 1222, 1178, 1034, 732, 718 cm⁻¹.

### Example 2b

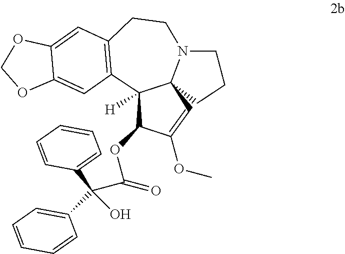

(−)-cephalotaxine 2,2-diphenylglycolate The OCA 1b was brought into contact with purified cephalotaxine according to the general procedure of example 2. The cephalotaxine ester 2b thus obtained has the following characteristics:

Physical state: white powder.

¹H NMR (300 MHz, CDCl₃) δ 7.27-7.10 (m, 8H), 7.01-6.87 (m, 2H), 6.64-6.54 (m, 1H), 6.28 (s, 1H), 6.03 (dd, J=9.7, 0.7, 1H), 5.97 (d, J=1.5, 1H) 5.89 (d, J=1.5, 1H) 5.01 (s, 1H), 3.97 (s, 1H), 3.79 (d, J=9.7, 1H), 3.64 (s, 1H), 3.03 (d, 1H), 2.86-2.68 (m, 1H), 2.63-2.48 (m, 2H), 2.50-2.32 (m, 1H), 2.09-1.59 (m, 5H); ¹³C NMR (75 MHz, CDCl₃) δ 174.04, 146.83, 145.87, 142.50, 141.29, 133.57, 127.96, 127.87, 127.74, 127.36, 112.65, 110.52, 101.02, 100.58, 81.23, 77.06, 57.40, 55.94, 53.92, 48.54, 43.34, 31.14, 29.99, 20.39; IR (KBr) n 3487, 2794, 1731, 1655, 1487, 1163, 1059, 1027 cm⁻¹.

### Example 2c

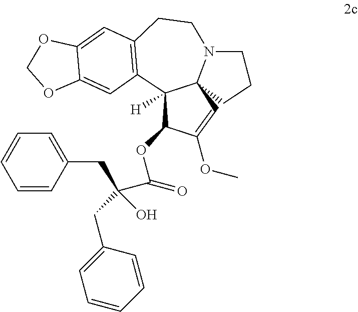

(−)-cephalotaxine 2,2-dibenzylglycolate The OCA 1c was brought into contact with purified cephalotaxine according to the general procedure of example 2. The cephalotaxine ester 2c thus obtained has the following characteristics:

Physical state: white powder.

¹H NMR (300 MHz, CDCl₃) δ 7.31-7.18 (m, 8H), 7.04-6.98 (m, 2H), 6.71 (s, 1H), 6.65 (s, 1H), 5.92-5.85 (m, 2H), 5.78 (d, J=1.4, 1H), 3.84 (s, J=9.6, 1H), 3.73 (s, 3H), 3.42-3.25 (m, 1H), 3.21-3.09 (m, 1H), 3.10-2.95 (m, 1H), 2.89 (d, J=13.6, 1H), 2.78 (s, 1H), 2.71 (d, J=13.6, 2H), 2.52 (dd, J=14.1, 6.7, 1H), 2.43 (d, J=13.6, 1H), 2.26 (d, J=13.6, 1H), 2.17-2.01 (m, 1H), 2.01-1.86 (m, 1H), 1.88-1.72 (m, 3H), 1.38-1.24 (m, 2H), 1.00 (d, J=6.6, OH), 0.96-0.84 (m, 2H); ¹³C NMR (75 MHz, CDCl₃) δ 175.24, 146.46, 136.28, 136.04, 131.02, 130.84, 128.29, 127.01, 113.40, 110.38, 101.30, 77.82, 76.02, 57.70, 56.41, 54.23, 44.32, 43.60, 32.00, 23.07, 20.68, 14.55; IR (ATR, film, neat) ν1731, 1652, 1484, 1452, 1366, 1339, 1267, 1096, 1035, 984, 930 cm⁻¹.

### Example 2d

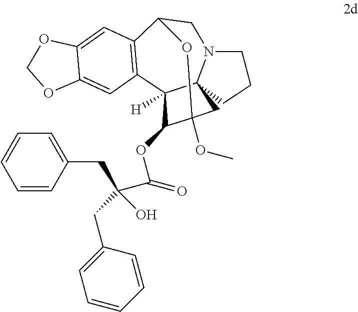

(−)-drupacine 2,2-dibenzylglycolate The OCA 1c was brought into contact with purified drupacine according to the general procedure of example 2. The drupacine ester 2d thus obtained has the following characteristics:

Physical state: white powder.

¹H NMR (300 MHz, CDCl₃) δ 7.35-7.18 (m, 10H), 6.73 (s, 1H), 6.55 (s, 1H), 5.87 (s, 1H), 5.87 (s, 1H), 5.65 (d, J=1.4, 1H), 5.00 (d, J=3.9, 1H), 4.83 (d, J=9.3, 1H), 3.73 (d, J=9.3, 1H), 3.55 (s, 3H), 3.16 (m, 4H), 2.91-, 2.77 (m, 3H), 2.60 (d, J=13.4, 1H), 2.47 (dd, J=17.5, 8.6, 1H), 2.34-2.19 (m, 3H), 2.15-2.01 (m, 1H), 1.91-1.73 (m, 3H), 1.60 (d, J=13.9, 1H); ¹³C NMR (75 MHz, CDCl₃) δ 175.23, 147.40, 146.49, 136.02, 130.91, 130.70, 128.16, 128.08, 126.75, 111.52, 108.12, 107.94, 101.27, 78.52, 76.14, 66.06, 57.17), 56.97, 54.20, 52.17, 43.43, 42.90, 35.99, 22.50; IR (ATR, film, neat) ν 2936, 1726, 1485, 1453, 1340, 1319, 1231, 1098, 1055, 1036, 930, 912 cm⁻¹.

### Example 2e

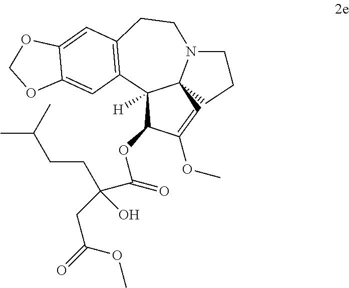 [SMILES: COC(=O)CC(O)(CCC(C)C)C(=O)O[C@@H]1C(OC)=C[C@]23CCCN2CCc2cc4c(cc2[C@H]13)OCO4]

Deoxyharringtonine* The OCA 1e was brought into contact with purified cephalotaxine according to the general procedure of example 2. The cephalotaxine ester 2e thus obtained has the following characteristics:

Physical state: colorless thick oil.

¹H NMR (300 MHz, CDCl₃) δ 6.61 (s, 1H), 6.53 (s, 1H), 5.98 (dd, 1H, J=10, 0.7 Hz), 5.85 (dd, 2H, J=11.8, 1.5 Hz), 5.03 (d, 1H, J=0.6 Hz), 3.76 (d, 1H, J=9.8 Hz), 3.66 (s, 3H), 3.56 (s, 3H), 3.47 (s, 1H), 3.11 (m, 2H), 2.92 (m, 1H), 2.56 (m, 2H), 2.36 (m, 1H), 2.26 (d, 1H, J=16 Hz), 2.03 (m, 1H), 1.90 (m, 1H), 1.87 (d, 1H, J=16 Hz), 1.73 (m, 2H), 1.40 (m, 3H), 1.27 (m, 1H), 0.96 (m, 1H), 0.83 (d, 3H, J=7.0 Hz), 0.82 (d, 3H, J=7 Hz)¹³C NMR (75 MHz, CDCl₃) δ 174.2, 170.5, 157.9, 146.7, 145.9, 133.4, 128.5, 12.7, 109.8, 100.9, 100.1, 74.8, 74.6, 70.7, 57.2, 55.9, 54.1, 51.6, 48.8, 43.5, 42.8, 36.8, 31.7, 31.4, 28.1, 22.8, 22.3, 20.4; IR (film, neat) ν 3500, 2955, 1748, 1653, 1504, 1488, 1225, 754 (m).

*This product is contaminated with 15% of epideoxyharringtonine.

### Example 2f

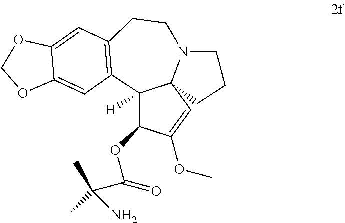

(−)-cephalotaxine 2-aminobutyrate The NCA 1g was brought into contact with purified cephalotaxine according to the general procedure of example 2. The cephalotaxine ester 2f thus obtained has the following characteristics:

Physical state: pale yellow powder.

¹H NMR (300 MHz, CDCl₃) δ 6.61 (s, 1H), 6.58 (s, 1H), 5.92-5.78 (m, 4H), 3.80 (d, J=9.7, OH), 3.71 (s, 1H), 3.21-3.04 (m, 1H), 3.01-2.90 (m, OH), 2.71-2.56 (m, 1H), 2.12-1.69 (m, 2H), 1.20 (s, 3H), 0.81 (s, 3H).

### Example 3

### Preparation of Cephalotaxyl Substituted Glycolate or Malate Esters of General Formula Via Bistrifluoro-Methylacetonide Intermediates

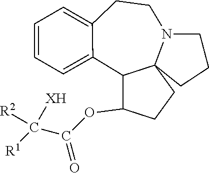

To a 0.5 M stirred dichloromethane solution, at room temperature, under argon, of a cephalotaxine was added a 0.5 M solution of the substituted bis-trifluoromethyl-lactone, one cristal of N,N-dimethyl-aminopyridine (DMAP) as catalyst. The disappearance of the cephalotaxine was checked by TLC. Work-up was completed by washing (water), drying (magnesium sulfate) and evaporating under reduce pressure. The crude cephalotaxine ester was purified by high resolution liquid chromatography on Silicagel Si60 using a gradient of methanol in dichloromethane. Monitoring of the fractionation was performed by thin layer chromatography. After evaporation of the selected fractions (TLC), the cephalotaxine ester was obtained as white solid.

### Example 3a

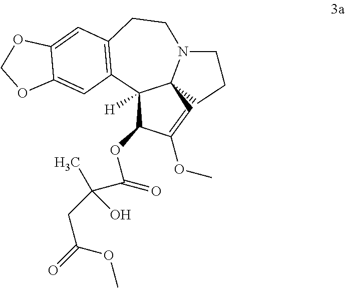 [SMILES: COC(=O)CC(C)(O)C(=O)O[C@@H]1C(OC)=C[C@]23CCCN2CCc2cc4c(cc2[C@H]13)OCO4]

Methyl, cephalotaxyl 2′-hydroxy-2′-methyl-succinate.* A solution of the lactone 1h was mixed with the solution of purified cephalotaxine, according the general operating procedure of example 3. The crude cephalotaxine ester is purified according the general procedure of the example 3 (yield 67%) This cephalotaxine ester 3a exhibited the following features:

Physic state: thick colorless oil

¹H NMR (300 MHz, CDCl₃) δ 6.60 (s, 1H), 6.54 (s, 1H), 6.00 (dd, 1H, J=9.5, 0.6 Hz), 5.80 (dd, 2H, J=11.8, 1.5 Hz), 5.01 (d, 1H, J=0.6 Hz), 3.76 (d, 1H, J=9.8 Hz), 3.66 (s, 3H), 3.56 (s, 3H), 3.47 (s, 1H), 3.11 (m, 2H), 2.92 (m, 1H), 2.54 (m, 2H), 2.32 (m, 1H), 2.24 (d, 1H), 1.70 (m, 2H), 1.40 (m, 3H), 0.96 (m, 1H), 0.83 (3H, s),

### Example 3b

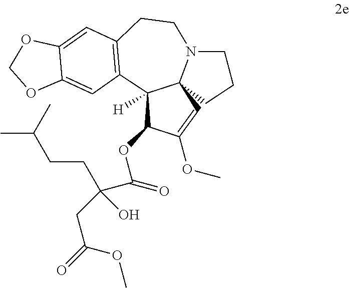 [SMILES: COC(=O)CC(O)(CCC(C)C)C(=O)O[C@@H]1C(OC)=C[C@]23CCCN2CCc2cc4c(cc2[C@H]13)OCO4]

Desoxyharringtonine.* A solution of the above described lactone 1i was mixed with the solution of purified cephalotaxine, according the general operating procedure of example 2B. The crude cephalotaxine ester was purified according the general procedure of example 3 (yield 76%) This cephalotaxine ester 2e (see example 2e) exhibited the following features:

Physic state: thick colorless oil

¹H NMR (300 MHz, CDCl₃) δ 6.61 (s, 1H), 6.53 (s, 1H), 5.98 (dd, 1H, J=10, 0.7 Hz), 5.85 (dd, 2H, J=11.8, 1.5 Hz), 5.03 (d, 1H, J=0.6 Hz), 3.76 (d, 1H, J=9.8 Hz), 3.66 (s, 3H), 3.56 (s, 3H), 3.47 (s, 1H), 3.11 (m, 2H), 2.92 (m, 1H), 2.56 (m, 2H), 2.36 (m, 1H), 2.26 (d, 1H, J=16 Hz), 2.03 (m, 1H), 1.90 (m, 1H), 1.87 (d, 1H, J=16 Hz), 1.73 (m, 2H), 1.40 (m, 3H), 1.27 (m, 1H), 0.96 (m, 1H), 0.83 (d, 3H, J=7.0 Hz), 0.82 (d, 3H, J=7 Hz)¹³C NMR (75 MHz, CDCl₃) δ 174.2, 170.5, 157.9, 146.7, 145.9, 133.4, 128.5, 12.7, 109.8, 100.9, 100.1, 74.8, 74.6, 70.7, 57.2, 55.9, 54.1, 51.6, 48.8, 43.5, 42.8, 36.8, 31.7, 31.4, 28.1, 22.8, 22.3, 20.4; IR (film, neat) ν 3500, 2955, 1748, 1653, 1504, 1488, 1225, 754 (m).

*The compound is contaminated with small amount of 2′-epideoxyharringtonine.

### Example 3c

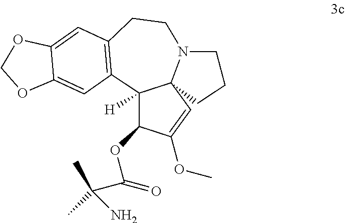

(−)-cephalotaxine 2-aminobutyrate The lactone 1j was brought into contact with purified cephalotaxine according to the general procedure of example 3. The cephalotaxine ester 2f thus obtained has the following characteristics:

Physical state: pale yellow powder.

¹H NMR (300 MHz, CDCl₃) δ 6.61 (s, 1H), 6.58 (s, 1H), 5.92-5.78 (m, 4H), 3.80 (d, J=9.7, OH), 3.71 (s, 1H), 3.21-3.04 (m, 1H), 3.01-2.90 (m), 2.71-2.56 (m, 1H), 2.12-1.69 (m, 2H), 1.20 (s, 3H), 0.81 (s, 3H).

### Example 3d

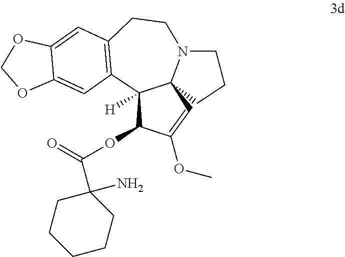 [SMILES: COC1=C[C@]23CCCN2CCc2cc4c(cc2[C@@H]3[C@@H]1OC(=O)C1(N)CCCCC1)OCO4]

(−)-Cephalotaxine 1-aminocyclohaxane carboxylate The lactone 1k was brought into contact with purified cephalotaxine according to the general procedure of example 3. The cephalotaxine aminoester 3d thus obtained has the following characteristics:

Physical state: amorphous white powder.

¹H NMR (300 MHz, C₆D₆+CDCl₃) δ 6.42 (s, 1H), 6.40 (s, 1H), 5.80-5.72 (m, 4H), 3.60 (d, J=9.7, 1H), 3.50 (s, 1H), 3.06-2.98 (m, 1H), 2.95-2.80 (m 3H), 2.60-2.40 (m, 5H), 2.05-1.50 (m, 3H).

1. A process for preparing cephalotaxine esters corresponding to the following general formula I which comprises the cephalotaxine backbone: 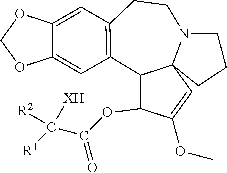
that can also be written as C(R¹)(R²)(XH)COO[CTX]
wherein CTX represents the cephalotaxine backbone, being optionally substituted,
in which formula I, X is a heteroatom, preferably an oxygen, a sulfur or a nitrogen, R¹ and R², taken separately, may be alkyl, cycloalkyl, heteroalkyl, aryl, heteroaryl, heterocycloalkyl or aralkyl groups, said groups being optionally interrupted by ester functions, or groups that can form one or more rings or a heterocycle together,
consisting in bringing the corresponding cephalotaxine compound, or salts, isomers or tautomeric forms thereof, which is free or which is in the form of a metal alkoxide CTXOM,
wherein CTX represents the cephalotaxine backbone, being optionally substituted,
in which M is a hydrogen atom or a metal atom,
into contact with a heterocyclic side chain precursor having both a bifunctional protected (bidentate) and activated (acylating) form of an acid bearing a hydrogenated heteroatom, in the alpha (α) position with respect to the carboxyl group, and corresponding to the following general formula: 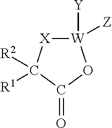
in which case W is a carbon, sulfur, silicon or bore atom, X, R¹ and R² have respectively the same meaning as above, it being possible for R¹ and R² to form a ring or a heterocycle together, and Y and Z are alkyl or heteroalkyl radicals, or monovalent heteroatoms, which may be identical or different, in an independent manner, or may fuse so as to give a divalent heteroatom, it being possible for the X—W bond to be covalent or ionic,
in a customary aprotic solvent, preferably with a catalyst which may be a hindered tertiary amine, at a temperature of between −80° C. and +100° C., preferably in the range 0 to 30° C.

2. The process of claim 1, wherein X is selected from an oxygen atom, a hydronitrogen (NH) pseudo atom and a sulfur atom.

3. The process of claim 1, in which W is a carbon atom.

4. The process according to claim 1, wherein Y, Z together fuse to give an oxygen atom.

5. The process according to claim 1, in which Y, Z are each an electro-attractive hetero-hydrocarbon group.

6. The process according to claim 1, in which Y, Z are identical and are trifluoromethyl.

7. The process of claim 1, in which W is a sulfur atom, X is an oxygen atom and Y, Z together fuse to give an oxygen atom.

8. The process of claim 1, in which W is a silicium atom, X is an oxygen atom and Y, Z are alkyl, aryl or aralkyl group.

9. The process of claim 2, in which W is a silicium atom, X is an oxygen atom and Y, Z are alkyl, aryl or aralkyl group

10. The process according to claim 1, in which R¹ and R² are identical.

11. The process according to claim 1, in which R² is —CH—₂COO—R³ in which R³ is selected from alkyl, cycloalkyl, heteroalkyl, aryl, heteroaryl, heterocycloalkyl and aralkyl groups, said groups being optionally interrupted by ester functions.

12. The family of compounds having the structural formula I which comprises the cephalotaxine backbone: 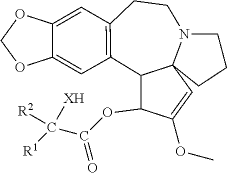
that can also be written C(R¹)(R²)(XH)COO[CTX]
wherein CTX represents the cephalotaxine backbone, being optionally substituted,
in which X is a heteroatom, preferably an oxygen, a sulfur or a nitrogen, R¹ and R², taken separately, are selected from alkyl, cycloalkyl, heteroalkyl, aryl, heteroaryl, heterocycloalkyl and aralkyl groups and groups that can form one or more rings or a heterocycle together,
with the proviso when together X═O and R¹═—CH₂COO—R³, R² is not (CH₃)₂(CH₂)ₙ— (with n=1 to 4) or R² is not (CH₃)₂COH(CH₂)ₙ₋₁— or R² is not benzyl or phenyl or homobenzyl, R³ is selected from alkyl, cycloalkyl, heteroalkyl, aryl, heteroaryl, heterocycloalkyl and aralkyl groups, said groups being optionally interrupted by ester functions.

13. The family of compounds of claim 12 wherein R¹ and R² are identical.

14. The family of compounds of claim 13, in which X is an oxygen atom.

15. The family of compounds of claim 13, in which X is an nitrogen atom.

16. The family of compounds of claim 13, in which X is an sulfur atom.

17. The family of compounds of claim 12
in which X is a nitrogen atom, R¹ and R² are different each other and taken separately, are selected from alkyl, cycloalkyl, heteroalkyl, aryl, heteroaryl, heterocycloalkyl and aralkyl groups and groups that can form one or more rings or a heterocycle together.

18. The compounds of claim 12, wherein the tetracyclic core namely the alkaloid moiety is the natural enantiomeric form of cephalotaxine, the latter having the below structural formula

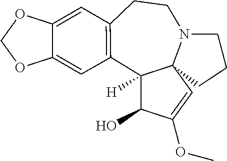 [SMILES: COC1=C[C@]23CCCN2CCc2cc4c(cc2[C@@H]3[C@@H]1O)OCO4]

19. The compounds of claim 12 in which the alkaloid moiety is the unnatural enantiomer.

20. The subset of compounds having the following structural formula 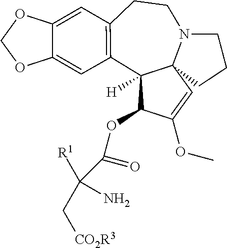
In which R¹ and R² have the same meaning as in claim 1 and R3 has the same meaning as R¹ or R².

21. A cephalotaxine ester of the family compounds of claim 12, said cephalotaxine ester being selected from the group consisting of:
(−)-cephalotaxine 2,2-dimethylglycolate, 2a
(−)-cephalotaxine 2,2-diphenylglycolate, 2b
(−)-cephalotaxine 2,2-dibenzylglycolate, 2c
(−)-drupacine 2,2-dibenzylglycolate, 2d
(−)-cephalotaxine 2-aminobutyrate, 2f
(−)-cephalotaxine 2-aminobutyrate, 3c, and
(−)-Cephalotaxine 1-aminocyclohaxane carboxylate, 3d.

22. A pharmaceutical preparation comprising one or more of the compounds depicted in claim 12.

23. A method of treating a disease selected from the group consisting of a human cancer including leukemia, parasitic disease, immune disease, and a transplantation rejection, the method comprising the step of administering an effective amount of the pharmaceutical preparation according to claim 22 to a subject in need thereof.

24. Compound as an intermediary compound for preparing cephalotaxine esters in accordance with the process as claimed in claim 1, said compound being selected from:
O-carboxyanhydride of diphenylglycolic acid, 1b
O-carboxyanhydride of dibenzyl glycolic acid, 1c
O-carboxyanhydride of primary dimethyl citrate, 1d
O-carboxyanhydride of deoxyharringtonic acid methyl hémiesters, 1e
O-carboxyanhydride of neoharringtonic acid methyl hémiesters, 1f
N-carboxyanhydride of dimethyl glycolic acid, 1g
O—O-Hexafluoroacetonide of methyl citramalic acid hémiesters, 1h
a substituted bis 5,5′-trifluoromethyl-1,4-dioxolanone, 1i
3,3′-dimethyl-5,5′-Bistrifluoromethyl-oxazolidinone, 1j
3,3′-cyclopentylidene-5,5′-Bistrifluoromethyl-oxazolidinone, 1k.
